(19) United States
(12) Patent Application Publication
(10) Pub. No.: US 2026/0351892 A1
(43) Pub. Date: Nov. 5, 2026

(54) METHODS AND SYSTEMS OF ELECTRICAL CONNECTOR ASSEMBLY FOR STRETCHABLE ELECTRONICS

(71) Applicant: Applied Cavitation, Inc., Goleta, CA (US)

(72) Inventors: Dana Lynn Hankey, Santa Barbara, CA (US); Marshall Campion Tibbetts, Goleta, CA (US); Andrew John Bambach, Goleta, CA (US)

(21) Appl. No.: 19/099,250

(22) PCT Filed: Jul. 31, 2023
(86) PCT No.: PCT/US2023/029156

§ 371 Date: Jan. 28, 2025

Related U.S. Application Data
(60) Provisional application No. 63/393,690, filed on Jul. 29, 2022.

(51) Int. Cl. H01R 13/52; H01R 13/512; H01R 43/26; H05B 3/34; H05K 1/02; H05K 1/16

(52) U.S. Cl. CPC H01R 13/5202; H01R 13/512; H01R 43/26; H05B 3/34; H05K 1/0212; H05K 1/0283; H05K 1/167; H05B 2203/003; H05K 2201/09263

(57) ABSTRACT

A system may include an electrical connector assembly that may include a flexible or stretchable substrate that may include an electrode. The assembly may include a watertight housing having a top member and a bottom member. The assembly may include an electrical connector disposed in the top member, the bottom member, or both and capable of conducting about two amperes of current when connected to an external power source. The assembly may include a coupling mechanism to couple the top member to the bottom member such that, when coupled, the electrode on the flexible or stretchable substrate is at least one of in communication or coupled to the electrical connector. The substrate can include a heating element and the electrical connector assembly can have an ambient temperature or a temperature below a temperature of the heating element when the external power source is in an operative state or when the assembly is used by a user.

### CROSS-REFERENCE TO RELATED PATENT APPLICATIONS

This application claims the benefit of and priority to U.S. Provisional Patent Application No. 63/393,690 filed Jul. 29, 2022, the entire disclosure of which is incorporated by reference herein.

### BACKGROUND

The present disclosure relates generally to the field of electronic connectors, wearables and wearable electronic components.

There are various types of connectors for electronic systems which can provide effective performance in normal and extreme use conditions. However, it is difficult to use currently available electrical connectors for reliably electrically coupling electronics provided on stretchable or flexible substrate materials to a rigid connector.

### SUMMARY

The described technology provides methods and materials that enable robust solutions for connectors to conduct current to the additively manufactured wearable electronics such as resistive heating elements. In one general aspect, electrical connector assembly may include an electrical connector assembly for an electrode on a flexible or stretchable substrate, the electrical connector assembly having a watertight housing having a top member and a bottom member. The electrical connector assembly may furthermore include an electrical connector disposed in at least one of the top member or the bottom member and capable of conducting about two amperes of current when connected to an external power source. The electrical connector assembly may in addition include a coupling mechanism to couple the top member to the bottom member such that, when coupled, the electrode on the flexible or stretchable substrate is at least one of in communication or coupled to the electrical connector.

Implementations may include one or more of the following features. When the electrical connector assembly is coupled with the electrode on the flexible or stretchable substrate, the electrode is at least one of in communication or coupled to the electrical connector. In some embodiments, the electrical connector assembly has an ambient temperature or a temperature below a temperature of the heating element during a period of time of the external power source being in an operative state. In some embodiments, the electrical connector assembly has an ambient temperature or a temperature below a temperature of the heating element when the electrical connector assembly is used by a user. The electrical connector assembly where the electrical connector assembly has an ambient temperature during charging or use for 1 minute to 24 hours. The electrical connector assembly where the coupling mechanism may include a nut and bolt connection. The electrical connector assembly where the coupling mechanism may include a magnetic connection. The electrical connector assembly where the magnetic connection may include magnets coupled to the substrate. The electrical connector assembly where the electrode on the stretchable or flexible substrate is electrically coupled to the coupling mechanism of the electrical connector assembly. The electrical connector assembly where the electrode on the stretchable or flexible substrate is electrically coupled to the coupling mechanism of the electrical connector assembly. The electrical connector assembly where the electrical connector further may include a plug and socket connection. The electrical connector assembly may include a configuration where the electrode on the flexible or stretchable substrate is disposed between the top member and the bottom member. In some embodiments, the connector can connect to the electrode printed on the substrate, e.g., the substrate that is, for example, has been heat pressed onto the fabric. The top and bottom members of the housing of the assembly can enclose the electrode on the substrate attached to the fabric, in some embodiments, the electrical connector assembly can be coupled to the electrode of the flexible or stretchable substrate from one side of the flexible or stretchable substrate. For example, the electrical connector assembly can be disposed on top of the flexible or stretchable substrate.

In one general aspect, a method for conducting current to an electrode on a flexible or stretchable substrate includes coupling an electrical connector assembly to the flexible or stretchable substrate, the electrical connector assembly where the electrical connector assembly may furthermore include a watertight housing, the watertight housing having a top member and a bottom member. The electrical connector assembly may in addition include an electrical connector disposed in at least one of the top member and the bottom member and capable of conducting about two amperes of current when connected to an external power source. Method may moreover include coupling, by a coupling mechanism, the top member to the bottom member such that, when coupled, the electrode on the flexible or stretchable substrate is at least one of in communication or coupled to the electrical connector.

The method may include coupling by a coupling mechanism, the top member to the bottom member such that, when coupled, the electrode on the flexible or stretchable substrate is at least one of in communication or coupled to the electrical connector. In some embodiments, the electrical connector assembly has an ambient temperature or a temperature below a temperature of the heating element during a period of time of the external power source being in an operative state. In some embodiments, the electrical connector assembly has an ambient temperature or a temperature below a temperature of the heating element when the electrical connector assembly is used by the user. The method where the electrical connector assembly has an ambient temperature or above ambient temperature during charging or use for 1 minute to 24 hours. The method where the coupling mechanism may include a nut and bolt connection. The method where the coupling mechanism may include a magnetic connection. The method where the magnetic connection may include magnets coupled to the substrate. The method where the electrode on the flexible substrate is electrically coupled to the coupling mechanism of the electrical connector assembly. The method where the electrode on the flexible or stretchable substrate is electrically coupled to the coupling mechanism of the electrical connector assembly. The method where the electrical connector further may include a plug and socket connection. The method where the electrode on the flexible or stretchable substrate is disposed between the top member and the bottom member.

In one general aspect, a system may include a flexible or stretchable substrate that may include an electrode. The system may furthermore include an electrical connector assembly having a watertight housing having a top member and a bottom member. The system may moreover include an electrical connector disposed in at least one of the top member and the bottom member and capable of conducting about two amperes of current when connected to an external power source. The system may also include a coupling mechanism to couple the top member to the bottom member such that, when coupled, the electrode on the flexible substrate is at least one of in communication or coupled to the electrical connector.

Implementations may include one or more of the following features. When the system is coupled, the electrode on the flexible or stretchable substrate is at least one of in communication or coupled to the electrical connector. In some embodiments, the substrate includes a heating element and the electrical connector assembly has an ambient temperature or a temperature below a temperature of the heating element during a period of time of the external power source being in an operative state. In some embodiments, the substrate includes a heating element and the electrical connector assembly has an ambient temperature or a temperature below a temperature of the heating element when the electrical connector assembly is used by a user. The system where the electrical connector assembly has an ambient temperature or above ambient temperature during charging or use for 1 minute to 24 hours. The system where the electrode on the flexible or stretchable substrate is electrically coupled to the coupling mechanism of the electrical connector assembly. The system where the electrode on the flexible or stretchable substrate is disposed between the top member and the bottom member. In some embodiments, a conductive interface can be used for electrically coupling the electrode of the substrate and the connector of the electrical connector assembly. In some embodiments, a conductive interface can be capable of being used in an electrical connection and capable of conducting about two amperes of current when connected to an external power source.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements. The drawings are for illustrative purposes and are not intended to limit the scope of the present disclosure.

FIG. 1 illustrates at least a partial view of an electrical connector assembly, according to some embodiments.

FIG. 2 illustrates a view of a housing of an electrical connector assembly where a top cover of the housing is spaced apart from a baseplate of the housing, according to some embodiments.

FIG. 3 illustrates an exploded view of an electrical connector assembly, according to some embodiments.

FIG. 4 illustrates results of an experiment measuring heat of the electrical connector assembly, according to some embodiments.

FIG. 5 illustrates a flexible or stretchable substrate having a resistive heater, according to some embodiments.

FIG. 6A illustrates a testing method measuring resistance of an electrode on a flexible or stretchable substrate when the flexible or stretchable substrate is in a stretched state, according to some embodiments.

FIG. 6B illustrates a testing method measuring resistance of an electrode on a flexible or stretchable substrate when the flexible or stretchable substrate is in a relaxed state.

FIG. 7 illustrates graph lines for resistance of an electrode on a flexible or stretchable substrate with respect to a plurality of stretch cycles, according to some embodiments.

FIGS. 8A-8B illustrate graph lines for resistance of an electrode on a flexible or stretchable substrate when the flexible or stretchable substrate is in a stretched state, and also a relaxed state, according to some embodiments.

FIGS. 9A-9B illustrate the heat output of an printed resistive heater on flexible or stretchable substrate before and after a plurality of wash cycles, according to some embodiments.

FIGS. 10A-10B illustrate graph lines for resistance of and power output of an electrode on a flexible or stretchable substrate depending on a number of wash cycles, according to some embodiments.

The details of various embodiments of the methods and systems are set forth in the accompanying drawings and the description below.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over, or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The techniques and materials described herein allow for the assembly of additively manufactured electronics, for example, resistive heating elements that can be heat pressed on, printed on, or included within or on materials used in items that benefit from the addition of heat, such as fabric elements, neoprene, leather, rubber, silicone, synthetic materials, clothing, seat covers, furniture, work gear, athletic gear, therapeutic items, medical items, wetsuits, blankets, and others. As used in this description, the term “printing” is intended to include all forms of printing and coating, including, but without limitation: pre-metered coatings such as patch die coating, slot or extrusion coating, slide or cascade coating, curtain coating; roll coating such as knife over roll coating, forward and reverse roll coating; gravure coating; dip coating; spray coating; meniscus coating; spin coating; brush coating; air knife coating; screen printing processes; electrostatic printing processes; thermal printing processes; ink jet printing processes; direct write printing processes, jettable deposition processes and other similar techniques.

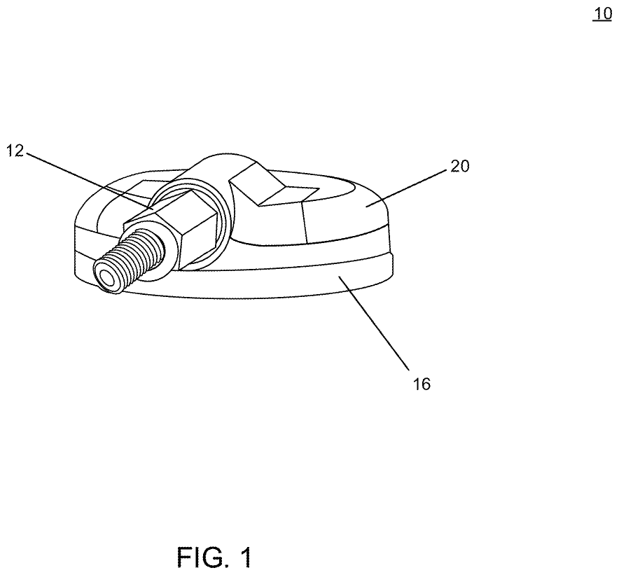

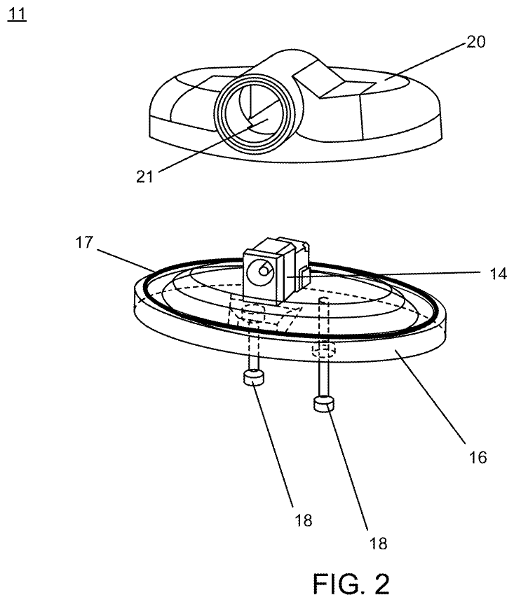

FIG. 1 illustrates at least a partial view of an electrical connector assembly 10 (or a mating assembly), according to some embodiments. The electrical connector assembly 10 can include a housing 11 (FIG. 2) that can include a top cover 20 and a baseplate 16. In some embodiments, the electrical connector assembly 10 can include a connector 12 (or a terminal connector, or an electrical connector) that can be mechanically coupled to the housing 11.

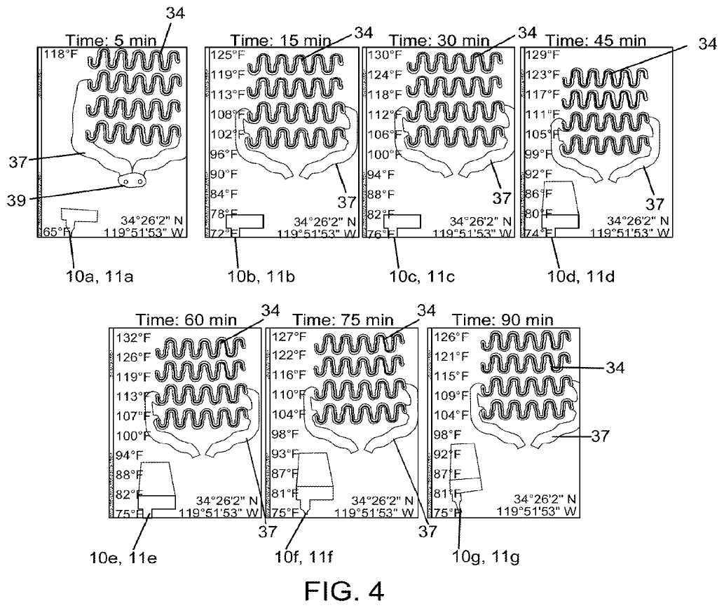

In some embodiments, the electrical connector assembly 10 can create a termination connection that can sustain rigorous mechanical, electrical, thermal, and/or environmental stresses that electrodes on flexible or stretchable substrates 36 (FIGS. 4-6), such as e-textiles, experience during their life cycle. In some embodiments, the present disclosure can enable higher current draw, e.g., equal or greater than 1 amp to provide sufficient power to stretchable electronics applications.

In some embodiments, the interface where the track terminations electrically couple to a control circuitry and/or a voltage source can be the points of mechanical and/or electrical failure. The housing 11 of the electrical connector assembly can be a multi-functioning component that shields the termination connection, mechanically fastens the connection, houses a terminal connector 12, and/or allows for a control circuitry (that can be, for example, an outside control circuitry) and/or a voltage source to electrically couple the voltage source to the electronics provided on the flexible or stretchable substrate 36 (FIGS. 4-6). The housing 11 can remain relatively fixed to the flexible or stretchable substrate 36 while being relatively easily replaceable in the event of failure.

FIG. 2 illustrates a view of the housing 11 of the electrical connector assembly 10 where the top cover 20 of the housing 11 is spaced apart from the baseplate 16, according to some embodiments. In one embodiment, and in brief overview, a baseplate 16 includes a jack 14 coupled to the baseplate 16. The baseplate 16 can typically be on one side of a flexible or stretchable substrate 36, with the jack 14 being electrically coupled with electrodes of the flexible or stretchable substrate 36 provided by the electronics on the flexible or stretchable substrate 36. The area where the jack 14 is disposed can be the area where the self-sealing screws 18 pass through the flexible or stretchable substrate 36, according to some embodiments. The top cover 20 is on the other side of the flexible or stretchable substrate 36. When the electrical connector assembly 10 is disposed at the desired place, the two screws 18 can be tightened, and the O-ring 17 seals around the electrode on the flexible or stretchable substrate 36 of the printed terminals (not shown).

Still referring to FIG. 2, and in greater detail, the top cover 20 of the housing 11 can include a housing opening or coupling access 21 to provide a through passage to electrically couple, e.g., the printed terminals of the flexible or stretchable substrate 36 and external electrical components directly or indirectly coupled to the electrical connector assembly 10. In some embodiments, the baseplate 16 may be formed of any suitable material that provides a mechanical support for the jack 14 and facilitates mechanical coupling of the baseplate 16 to the flexible or stretchable substrate 36. For example, the baseplate 16 may be formed of plastic materials such as polyethylene terephthalate, high-density polyethylene, polyvinyl chloride, low-density polyethylene, and/or polypropylene. In other embodiments, the baseplate 16 may be formed of rubber and the like materials. In still other embodiments, the baseplate 16 may be formed from structural fabric, such as polyester, vinyl-coated polyester, vinyl-laminate polyester, fiberglass, and/or meshes woven from these materials.

In some embodiments, the jack 14 may receive a corresponding electrical connector 12 which facilitates electrical coupling to be formed between the external electrical components (e.g., providing or transmitting power) and the electrode on the flexible or stretchable substrate 36. In some embodiments, the jack 14 may receive the electrical connectors 12, that can be, for example, barrel plugs with center pins and cantilever springs, Electronic Industries Alliance of Japan (EIAJ) connectors, or Radio Corporation of America (RCA) type barrel plugs. In other embodiments, the jack 14 may receive the electrical connectors 12, that can be, for example, a Universal Serial Bus (USB) connector, e.g., USB-A, USB-C, or rectangular header plugs. In some embodiments, the electrical connector 12 can be a socket capable to receive a plug connected to the external electrical components (e.g., a power source).

As shown in FIG. 2, the watertight connectors 18 (or self-sealing screws) couple the baseplate 16 to a top cover 20. In some embodiments, although two self-sealing screws 18 are shown in the figure, any number of connectors can be used to secure the top cover 20 to the base plate 16. In other embodiments, alternate securing mechanisms are employed, such as pressure fit between the top cover 20 and the baseplate 16 or tabbed locking mechanisms.

As shown in FIG. 2, the baseplate 16 is on one side of the flexible or stretchable substrate 36. In some embodiments, the baseplate 16 couples the electrodes provided by the electronics on the flexible or stretchable substrate 36. In one implementation, a strain relief can be incorporated in the area of the flexible or stretchable substrate 36 where the printed electronic trace enters the watertight connector 18. The jack 14 and the watertight connectors 18 pass through the flexible or stretchable substrate 36. The top cover 20 is on the other (e.g., an opposite) side of the flexible or stretchable substrate 36. When the electrical connector assembly 10 is displaced in a proper place, the O-ring seal(s) 17 around the printed terminals of the substrate 36.

In some embodiments, the electrical connector assembly 10 encloses in a watertight casing a section of the flexible or stretchable substrate 36 (with a printed electronic trace on the flexible or stretchable substrate 36) and the rigid surface to which the flexible or stretchable substrate 36 connects. In some embodiments, such watertight casing may include a standard electrical connector and a watertight closure on the outside of the casing which facilitates a standard plug to electrically couple the external electrical components to the flexible or stretchable substrate 36. For example, a standard electrical connection can facilitate dielectric coupling of elements of the electronic system external or outside the watertight casing. During an operative state (when, e.g., in active connection with the electrode on the flexible or stretchable substrate 36), the control circuit and battery can be plugged into the electrical connectors 12. During an inoperative state, the control circuit and battery are unplugged, and, to reduce or protect the components disposed within the housing 11 from dust and water, a watertight plug or seal (not shown) may be used to seal the connection or coupling access 21, so that the coupling access 21is watertight.

In some embodiments, the electrical connector 12 has a connector cover (not illustrated) to cover the electrical connector 12. When the electrical connector assembly is not charged and/or not coupled to the external control circuit and battery, the connector cover may cover the electrical connector 12, to protect from dust and water. For example, the connector cover can enclose the exposed end of the body of the electrical connector 12 such that interfaces between the connector cover, the body of the electrical connector 12, and the housing 11 are watertight.

In some embodiments, the connector in FIG. 1 can connect to the electrode printed on the substrate 36, the substrate 36 that, e.g., has been heat pressed onto the fabric 76. Though the top and bottom members 19 and 24, respectively, in FIG. 2 can enclose the electrode on the substrate 36 attached to the fabric 76, in some embodiments, the electrical connector assembly 10 can be coupled to the electrode of the flexible or stretchable substrate 36 from one side of the flexible or stretchable substrate 36. For example, the electrical connector assembly 10 can be disposed on top of the flexible or stretchable substrate 36.

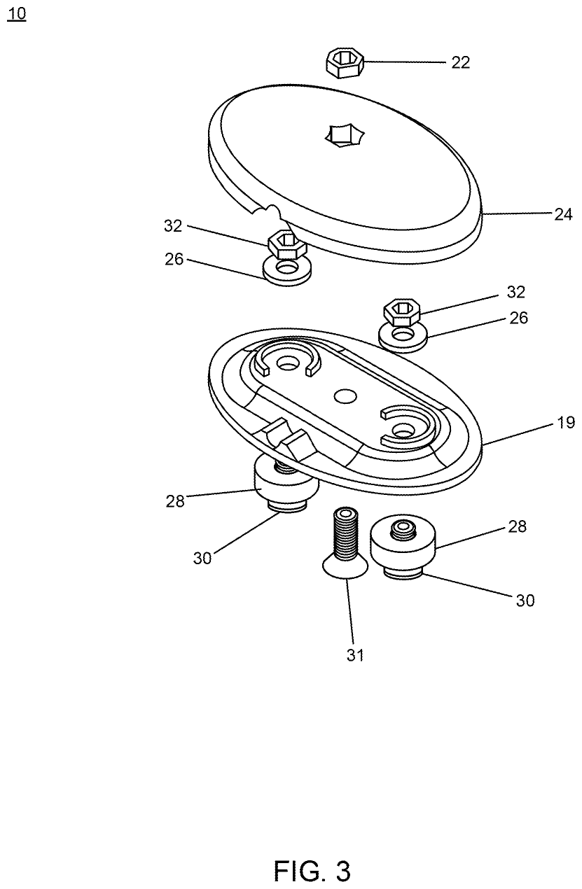

In some embodiments, a connector may be coupled to the circuitry of the flexible or stretchable substrate 36 using magnetic force rather than or in combination with the physical force. For example, referring to FIG. 3, and in brief overview, an electrical connector assembly 10 (or a mating assembly) is illustrated, according to some embodiments. The mating assembly 10 can include a top component 24, a bottom component 19, two ring magnets 28 connected to the bottom component by screws 30, nuts 32 and washers 26. The top component 24 is coupled with the bottom component 19 using a screw and nut (e.g., a screw 31 and nut 22) or the like arrangement.

Still referring to FIG. 3, and in greater detail, the mating assembly 10 includes a top component 24 and a bottom component 19. The top component 24 and bottom component 19 may be formed of any suitable material. For example, the top component 24 and bottom component 19 may be formed of plastic materials such as polyethylene terephthalate, high-density polyethylene, polyvinyl chloride, low-density polyethylene and/or polypropylene. In other embodiments, the top component 24 and the bottom component 19 may be formed of rubber. In still other embodiments, the top component 24 and the bottom component 19 may be formed of structural fabric, such as polyester, vinyl-coated polyester, vinyl-laminate polyester, fiberglass, and/or meshes woven from these materials. In some embodiments, the top component 24 and the bottom component 19 may be formed from different materials.

The mating assembly 10 can have two ring magnets 28 that couple or mate with corresponding ring magnets provided by the flexible or stretchable substrate 36. For example, the ring magnets 28 may be relatively permanent magnets, such as Neodymium Iron Boron, Samariam Cobalt, Alnico, and/or ceramic or ferrite magnets. As shown in the FIG. 3, the ring magnets 28 are coupled to the mating assembly 10 using the screws 30, the nuts 32, and the washers 26. In this embodiment, conductive elements (that are, e.g., electrically coupled to the printed electronic trace of the flexible or stretchable substrate 36) external to the electrical connector assembly 10 and the flexible or stretchable substrate 36 may be wrapped around the screw 30 and held in place by pressure from the nut 32 and the washer 26. In other embodiments, the ring magnet 28 may be coupled to the mating assembly 10 using an adhesive and/or electrical coupling. In still other embodiments, the bottom component 19 may provide appropriately sized and shaped recess that allow the ring magnets 28 to be retained in the recess by pressure. In addition to magnetically coupling the mating assembly 10 to the flexible or stretchable substrate 36, the ring magnets 28 facilitate electrical coupling between the external circuitry and the circuitry provided by the flexible or stretchable substrate 36.

As illustrated in FIG. 3, the top component 24 is assembled with the bottom component 19 using a screw and nut arrangement (e.g., the screw 31 and the nut 22). In other embodiments, alternate securing mechanisms are employed, such as pressure fit between the top component 24 and the bottom component 19 or a tabbed locking mechanism.

The corresponding ring magnets 28 provided by the flexible, flexible or stretchable substrate 36 may be provided separately or as part of an assembly that includes the mating assembly 10. In some embodiments, the magnets may be sewn into fabric or affixed to the flexible or stretchable substrate 36 using an adhesive. In particular embodiments, the circuitry provided by the flexible or stretchable substrate 36 may have a terminal that is mechanically coupled between a ring magnet provided by the flexible or stretchable substrate 36 and a ring magnet 28 provided by the mating assembly 10.

In some embodiments, a conductive interface can be used for electrically coupling the electrode of the substrate 36 and the connector 12 of the electrical connector assembly 10. In some embodiments, a conductive interface can be capable of being used in an electrical connection and capable of conducting about two amperes of current when connected to an external power source. In some embodiments, the electrical connector assembly 10 can be electrically coupled on one side, e.g., on the top, of the substrate 36, e.g., in the area where a linkage element 39 (FIG. 4) is disposed. In some embodiments, the connector 12 can be electrically coupled to a power source through, e.g., a wire that is terminated in the connector 12. For example, the connector 12 can rely, at least partially, on a conductive interface to transfer the current from the incoming power source (e.g., through the wire). In some embodiments, the heater 34 that has the connection point of the linkage element 39 on the top of the substrate 36 where the connector 12 can press against the right and left termination of the heater 34.

FIG. 4 illustrates results of an experiment measuring heat of the electrical connector assembly 10a-10g (or the mating assembly), that have the housings 11a-11g. For example, the mating assembly 10a-10g having the magnetic connector of the type described herein was used to electrically couple a 5.1 Volt power source to a heating element 34 having a resistance of 2.4 Ohms, resulting in 2.1 Amps current and 10.7 Watts of power. The heating element 34 is coupled to the conducting elements 37 that are coupled to a linkage element 39. The linkage element 39 is coupled to the mating assembly 10. Heat emitted by the heating element 34 was measured using infrared imaging techniques at five minutes, fifteen minutes, 30 minutes, 45 minutes, 60 minutes, 75 minutes and 90 minutes from the moment when current starts conducting from the power source. Images of those measurements are shown in FIG. 4, according to some embodiments.

The experiment illustrated in FIG. 4, shows that the electrical connector assembly 10 supplied sufficient power to the heating element 34 to maintain a temperature in excess of 110 degrees Fahrenheit for at least 90 minutes. Further, observation of the infrared images shows that the connector housing 11a-11g remained at an ambient temperature, showing the electrical coupling was sufficiently formed to avoid or substantially reduce heat buildup over time. In some embodiments, the electrical connector assembly 10 has an ambient temperature or a temperature below a temperature of the heating element 34 during a period of time of the external power source being in an operative state. In some embodiments, the electrical connector assembly 10 has an ambient temperature or a temperature below a temperature of the heating element 34 when the electrical connector assembly 10 is used by a user. For example, the electrical connector assembly 10 can have an ambient temperature, during charging or use for 1 minute to 24 hours. In some embodiments, use time can depend on the external power source (e.g., a battery). In some embodiments, the heater 34 can be used at very low power for longer periods of time in comparison to when the heater 34 runs at hotter temperatures.

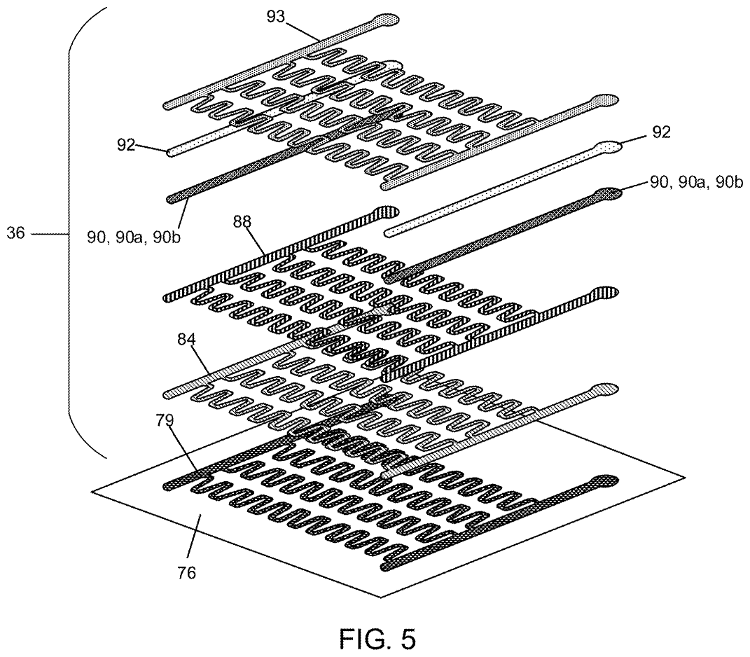

FIG. 5 illustrates an embodiment of an electrical structure 36 (or the flexible or stretchable substrate) providing a resistive heater. In some embodiments, the resistive heater that can be, for example, fixed and/or stretch-tolerant. As shown in FIG. 5, and in brief overview, the electrical structure 36 can include a plurality of layers. For example, the electrical structure 36 can include a base thermoplastic polyurethane (TPU) film 84 that can be disposed on, for example, an adhesive layer 79 that is spread over a fabric 76. A fixed resistance stretchable heater ink 88 (or heating element) is disposed on the TPU layer 84 and a stretchable printed silver conductor 90 is disposed at least partially over the heater ink 88.

In some embodiments, the heating element 88 is covered using a stretchable insulator ink 93.

Still referring to the FIG. 5, and in greater detail, the base TPU film 84 may be printed onto the fabric 76 and customized to satisfy the performance requirements for the smart textile material used for various applications. For example, TPUs 84 extruded with polyester provide resistance to chemicals and oils while materials extruded with polyether TPUs 84 offer flexibility and tear resistance. In some embodiments, TPUs 84 extruded with polycaprolactone are hydrolysis resistant are useful for applications with prolonged water exposure. In some embodiments, the TPU film 84 may include metals to provide a metallic color to the fabric. In still other embodiments, the TPU film 84 may include various materials to enhance antistatic properties.

In some embodiments, the resistive elements 88 can be disposed on the TPU film 84. In some embodiments, the resistive elements are printed over the TPU film 84. In one embodiment, the resistive elements are printed using materials such as SE5025, that is a stretchable, resistive ink manufactured by Applied Cavitation Inc. of Goleta, California. In some embodiments, the resistive elements such as SE5025 is designed for heating applications integrated onto elastomeric substrates. When cured, the ink such as the SE5025 material has a set resistance value and offers enhanced flexibility and stretchability. In some embodiments, the SE5025 material provides an enhanced adhesion to the TPU layer 84 and other elastomeric substrates.

The conductive component 90 can include silver traces 90a and/or a busbar system 90b. In some embodiments, the conductive component 90 can be disposed at least partially over the resistive elements 88. In some embodiments, the silver traces 90a and the busbar system 90b are printed on the resistive elements 88. In some embodiments, a stretchable silver conductor can be disposed on the TPU film 84. For example, the silver conductor is printed over the TPU film 84. In one embodiment, the silver conductor elements, such as the traces 90a and the busbar system 90b elements, are printed using an ink such as the SE1109 material. The conductive component material can be a stretchable silver ink such as SE1109 manufactured by Applied Cavitation Inc. of Goleta, California. In some embodiments, the conductive component material is a silver-filled conductor for printed interconnects for devices on elastomeric substrates. After drying, the ink has an enhanced conductivity and offers enhanced elongation and flexibility. In some embodiments, the SE1109 material has superior adhesion to thermoplastic urethane (TPU) materials. In some embodiments, the conductive component material can be used in stretchable electronics and e-textile applications to power components and/or devices and transmit signals from embedded devices and/or sensors.

In some embodiments, a stretchable carbon conductive element 92 can be disposed over the silver conductive elements 90. For example, the carbon elements 92 are printed on the silver conductive elements 92. In one embodiment, the carbon elements 92 are printed using an ink such as the SE1502 material, that is a stretchable, carbon conductor manufactured by Applied Cavitation Inc. of Goleta, California. In some embodiments, the SE1502 material is a carbon filled conductor for printed circuitry and/or devices on elastomeric substrates. The SE1502 material can be dried at low temperatures to accommodate sensitive substrates and devices. After curing, the ink has sufficient conductivity and offers enhanced elongation and flexibility. In some embodiments, the SE1502 material limits silver migration when applied over silver traces, e.g., over a conductive component 90.

In some embodiments, an insulator layer 93 is applied. In some embodiments, the insulator layer is printed at least partially on the carbon conductive elements 92. In some embodiments, an insulator layer 93 is disposed at least partially over the conductive silver traces 90a and the busbar system 90b. In some embodiments, the insulator layer 90 is printed on the silver traces 90a. In some embodiments, an insulator layer 93 is disposed at least partially over the resistive elements such as SE5025. In one embodiment, the insulator layer 90 is printed using an ink such as a SE3104 material. The SE3104 material is a stretchable, printable insulator manufactured by Applied Cavitation Inc. of Goleta, California. In some embodiments, the SE3104 material is a screen printable, thermally cured ink, and/or an ink cured with an ultraviolet (UV) light; the SE3104 material is stretchable when cured. In some embodiments, the SE3104 material can be used as an insulator and/or crossover dielectric.

When cured, the ink displays enhanced durability and flexibility, as well as high insulation resistance.

The electrical connector assembly 10 described herein can be utilized in various settings. For example, the electrical connector assembly 10 described above may be used in smart textiles to supply power for a circuitry provided by the smart textile. In some embodiments, the electrical connector assembly 10 described above may be used in smart textiles to transfer data. In some embodiments, a fixed resistance heater may be provided by a smart textile and powered by the current supplied by the electrical connector assembly 10.

The smart textiles providing fixed resistance heaters manufactured according to the above embodiment are useful in a variety of settings. For example, they can be used in outerwear, base layers, gloves, socks, and/or chairs for outdoor activities. Military applications of the smart textiles described herein include outerwear, gloves, seats, and/or other gear.

Therapeutic applications include braces, wraps, pads, bedding, and/or fabric(s) for pain management. Automotive applications include seat warming, steering wheel warming, defrosting applications, and/or warming of interior panels.

In some embodiments, the electrical connector assemblies 10 described herein may provide power for a smart textile providing electrical muscle stimulation. In some embodiments, an electrical structure 36 can provide electrical stimulation to muscle tissue.

Smart textiles providing fixed resistance heaters manufactured according to the above embodiment are useful in a variety of settings, for example, performance recovery, injury mitigation and/or pain management. An example of the electrical connector assemblies 10 described herein being used for data transfer is in a biometric sensor use case, where the printed wearable traces are used can be utilized for transferring data signals such as those used in electromyography (EMG), electrocardiogram (ECG/EKG), and/or electroencephalography (EEG). The electrical connector assembly 10 can be used for electrically coupling the biometric sensors and for transmitting current for electrical muscle stimulation (EMS).

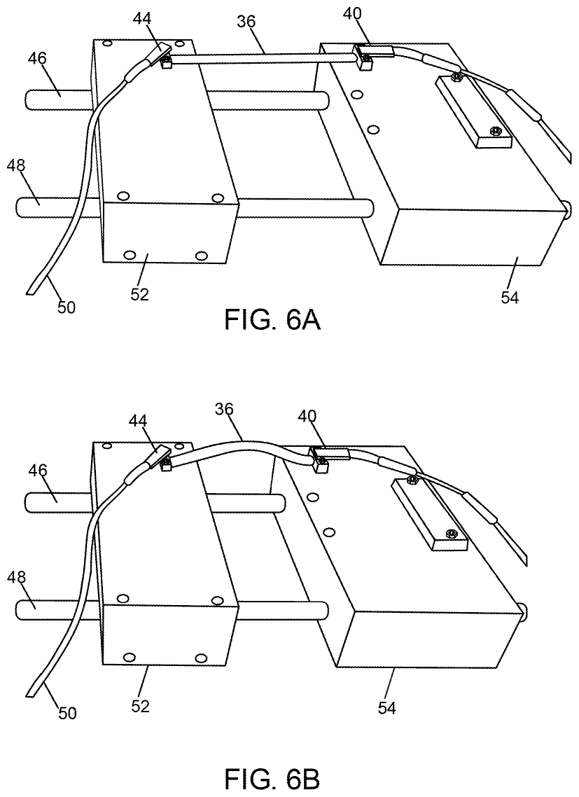

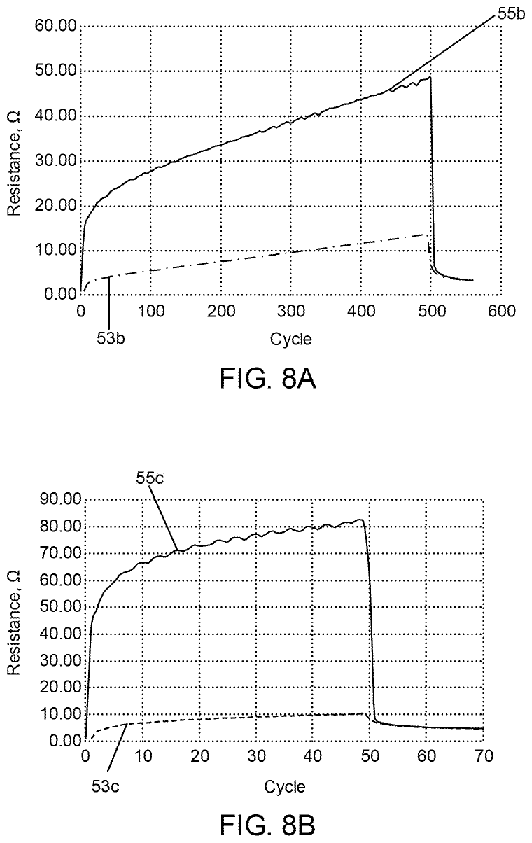

FIGS. 6A-10B illustrate the electrode on the flexible or stretchable substrate 36 maintaining the resistance when stretched and/or after a plurality of wash cycles. For example, FIGS. 6A-6B illustrate the electrode on the flexible or stretchable substrate 36 used in smart textiles fabricated as described herein that have enhanced performance with respect to maintaining resistance when the flexible or stretchable substrate 36 is stretched. FIGS. 8A-10B illustrate the electrode on the flexible or stretchable substrate 36 used in smart textiles fabricated as described herein and that can maintain resistance and electrical performance after multiple stretch or wash cycles. For example, various tests were performed to measure this performance and those results are provided below.

In FIG. 6A, a resistance of the printed conductor transferred onto fabric 36 was stretched and tested per cycle in Ohms. In FIG. 6B, a resistance of a printed conductor transferred onto fabric 36 (e.g., in a relaxed state) can be tested in a relaxed state per cycle in Ohms. The flexible or stretchable printed conductor transferred onto fabric 36 was tested on a stretch testing machine capable of controlling elongation, elongation rate, and a number of cycles. The stretch testing machine assembled by Applied Cavitation Inc. of Goleta, California. Also, KEITHLY 2110® (manufactured by TRS-RenTelco of Dallas, Texas) logging Benchtop digital multimeter having a 5.5 digit, up to 32 points per second was used for measurements. In some embodiments, digital calipers were used to verify elongation values.

The stretch testing machine may include a plurality of blocks 52, 54 that are slidably coupled to rails 46, 48. In some embodiments, a first end of the flexible or stretchable substrate 36 is coupled to one of the plurality of blocks 52, 54, e.g., block 52. A second end (e.g., an end opposite to the first end) of the flexible or stretchable substrate 36 is coupled to another block of the plurality of blocks 52, 54, e.g., block 54. Each end of the of the electrode on the flexible or stretchable substrate 36 is electrically coupled to clips 40, 44 that conduct the current through the electrode on the flexible or stretchable substrate 36 and transmit signals to the measurement module of the stretch testing machine. When one or both blocks 52, 54 slide on the rails 46, 48 in an opposite direction away from each other, the first and second ends of the flexible or stretchable substrate 36 move along with the blocks 52, 54 and can stretch the flexible or stretchable substrate 36. Signals corresponding to the resistance of the electrode on the flexible or stretchable substrate 36 in the stretched and relaxed states are transmitted to a controller of the stretch testing machine to be analyzed and presented on, e.g., a graphical user interface (GUI).

The following test samples of the printed conductor transferred onto flexible or stretchable substrate 36 can be used. The samples were printed with a 200.0016 Stainless Steel Mesh. Sample dimensions can be 76.2 mm in length by 2.0 mm in width (e.g., 38.1 Squares). The following tests can be performed: case 1 having the flexible or stretchable substrate 36 formed of the SE1109 material on 4-mil TPU; case 2 having the flexible or stretchable substrate 36 formed of the SE1109 material on 4-mil TPU with three layers and an insulator formed of the SE3104 material; case 3 having the flexible or stretchable substrate 36 formed of the SE1109 material on 4-mil TPU with three layers and the insulator formed of SE3104 material, bonded to fabric with, e.g., 3 mil hot-melt adhesive.

The following methods can be used for testing. Method 1 for cases 1 through 3 achieved 20% Elongation (15.2 mm displacement), 20% 1/s elongation rate (15.2 mm/s), 5000 cycles. Method 2 for case 1 achieved 50% Elongation (38.1 mm displacement), 20% 1/s elongation rate (15.2 mm/s), 500 cycles. Method 3 for case 1 achieved 100% Elongation (76.2 mm displacement), 20% 1/s elongation rate (15.2 mm/s), 50 cycles.

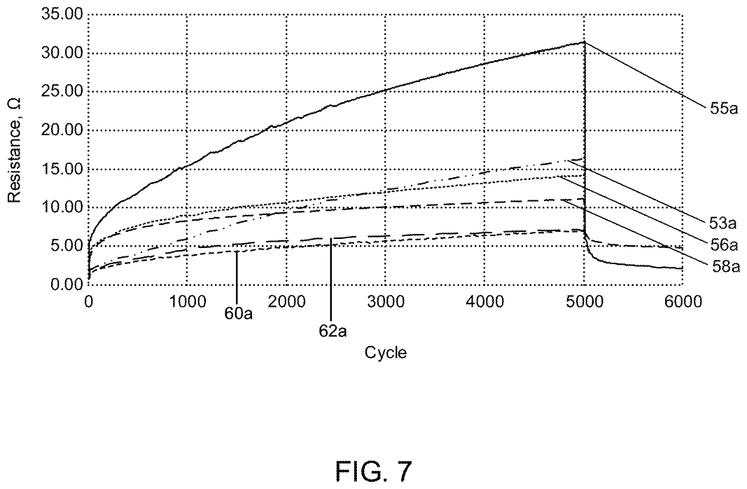

FIG. 7 illustrates a resistance (measured in Ohms) with respect to the number of stretch cycles when, for example, the flexible or stretchable substrate 36 can be 20% stretched, e.g., 20% 1/s elongation rate (15.2 mm/s), having 5000 cycles. In some embodiments, having the measured performance for 5000 cycles, a maximum resistance in the stretched condition for a printed conductor transferred onto flexible or stretchable substrate 36 formed of the SE1109 material on 4-mil TPU is illustrated by a curve 55a (or graph line). A lowest resistance for a printed conductor transferred onto a flexible or stretchable substrate 36 in a relaxed state is illustrated by a curve 53a (or graph line).

A maximum resistance in the stretched condition for a printed conductor transferred onto flexible or stretchable substrate 36 formed of the SE1109 material on 4-mil TPU having three layers and the insulator formed of the SE3104 material is illustrated by a curve 56a (or graph line). A lowest resistance for such flexible or stretchable substrate 36 at the relaxed state is illustrated by a curve 60a (or graph line).

A maximum resistance in the stretched condition for a printed conductor transferred onto flexible or stretchable substrate 36 having the flexible or stretchable substrate 36 formed of the SE1109 material on 4-mil TPU having three layers and the insulator formed of the SE3104 material, bonded to the fabric 76 with, e.g., 3 mil hot-melt adhesive is illustrated by a curve 58a (or graph line). A lowest resistance for such flexible or stretchable substrate 36 in the relaxed state is illustrated by a curve 62a (or graph line).

FIG. 8A illustrates a resistance (measured in Ohms) with respect to the number of stretch cycles when, for example, the printed conductor transferred onto flexible or stretchable substrate 36 can be 50% stretched, having 20% 1/s elongation rate (15.2 mm/s), 500 cycles. For example, a curve 55b (or graph line) shows a maximum resistance in the stretched state. For example, a curve 53b (or graph line) shows a lowest resistance in the relaxed state for such a printed conductor transferred onto flexible or stretchable substrate 36.

FIG. 8B illustrates a resistance (measured in Ohms) with respect to the number of stretch cycles when, for example, the printed conductor transferred onto flexible or stretchable substrate 36 can be 100% stretched, having 20% 1/s elongation rate (15.2 mm/s), having 50 cycles. For example, a curve 55c (or graph line) shows a maximum resistance in the stretched state. For example, a curve 53c (or graph line) shows a lowest resistance in the relaxed state for such flexible or stretchable substrate 36.

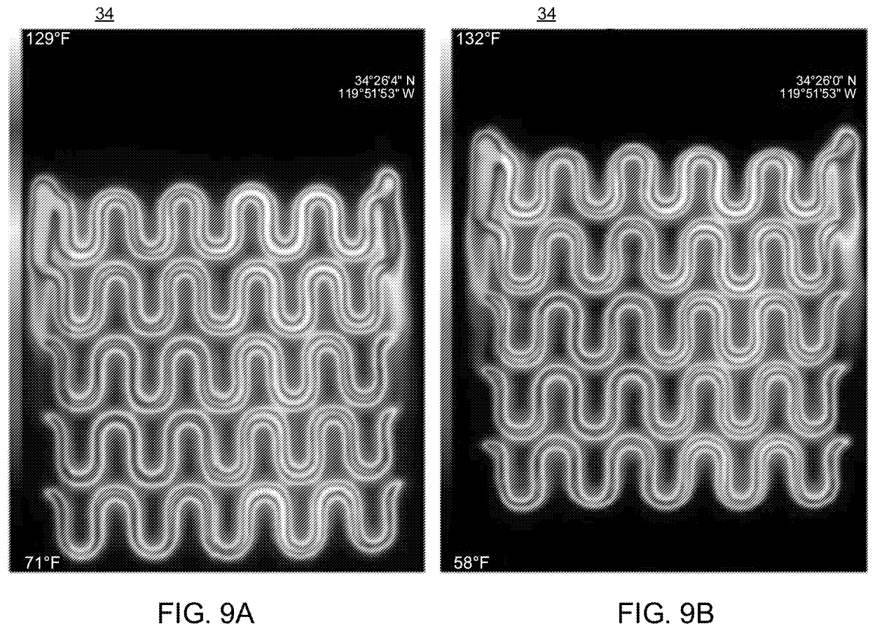

FIG. 9A and FIG. 9B illustrate at least a partial view of the heater 34 of the printed conductor transferred onto flexible or stretchable substrate 36 before and after a wash method, according to some embodiments. The following conditions can be set for testing: a front load washer, a delicate cycle having a laundry bag, a medium to low water temperature, an unscented liquid high efficiency (HE) laundry detergent, the flexible or stretchable substrate 36 can be washed with delicate and cotton items, hang dry technique was used. FIG. 9A illustrates at least a partial view of the heater 34 before the heater 34 can be washed. FIG. 9B illustrates at least a partial view of the heater 34 after the heater 34 had undergone fifteen wash cycles.

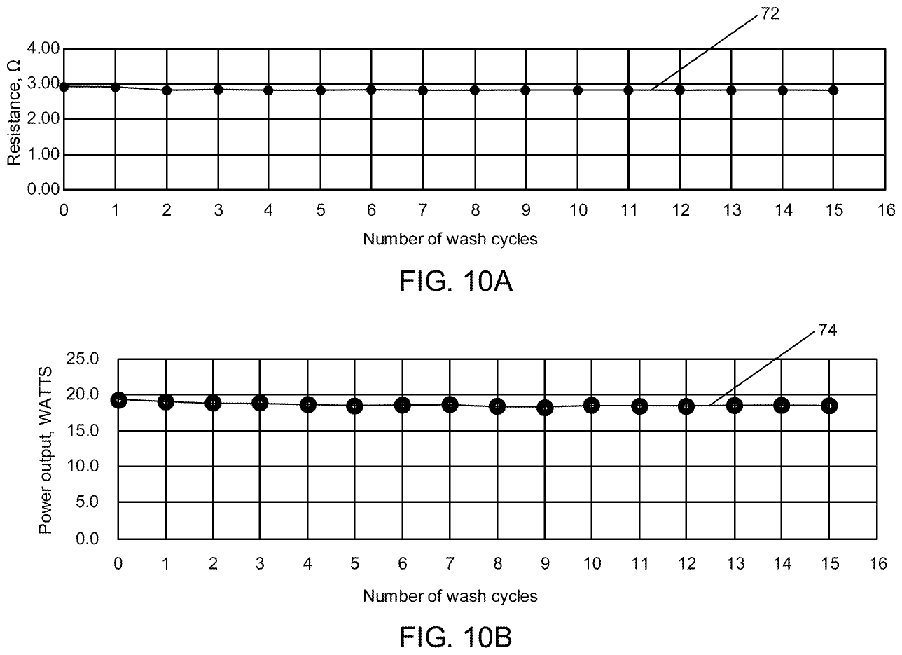

FIG. 10A illustrates a graph line 72 of resistance (in Ohms) of the heater 34 of the flexible or stretchable substrate 36 with respect to the number of wash cycles.

FIG. 10B illustrates a graph line 74 of power output (in Watts) of the heater 34 of the flexible or stretchable substrate 36 with respect to the number of wash cycles.

The term “coupled” and variations thereof includes the joining of two members directly or indirectly to one another. The term “electrically coupled” or “in communication” and variations thereof includes the joining of two members directly or indirectly to one another through conductive materials (e.g., metal or copper traces). Such joining (for both terms “coupled” and “electrically coupled”) may be stationary (e.g., permanent or fixed) or moveable (e.g., removable or releasable). Such joining (for both terms “coupled” and “electrically coupled”) may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

It should be noted that certain passages of this disclosure can reference terms such as “first” and “second” in connection with subsets of transmit spatial streams, sounding frames, response, and devices, for purposes of identifying or differentiating one from another or from others. These terms are not intended to merely relate entities (e.g., a first device and a second device) temporally or according to a sequence, although in some cases, these entities can include such a relationship. Nor do these terms limit the number of possible entities that can operate within a system or environment. It should be understood that the systems described above can provide multiple ones of any or each of those components and these components can be provided on either a standalone machine or, in some embodiments, on multiple machines in a distributed system.

While the foregoing written description of the methods and systems enables one of ordinary skill to make and use embodiments thereof, those of ordinary skill will understand and appreciate the existence of variations, combinations, and equivalents of the specific embodiment, method, and examples herein. The present methods and systems should therefore not be limited by the above described embodiments, methods, and examples, but by all embodiments and methods within the scope and spirit of the disclosure.

What is claimed is:

1. An electrical connector assembly for an electrode on a substrate, wherein the substrate is at least one of flexible or stretchable, the electrical connector assembly comprising:
a watertight housing; and
an electrical connector disposed in the watertight housing and capable of conducting about two amperes of current when connected to an external power source;
wherein the electrode on the substrate is at least one of in communication or coupled to the electrical connector.

2. The electrical connector assembly of claim 1,
wherein the substrate includes a heating element; and
wherein the electrical connector assembly has at least one of an ambient temperature or a temperature below a temperature of the heating element during at least one of (i) a period of time of the external power source being in an operative state or (ii) use of the electrical connector assembly by a user.

3. The electrical connector assembly of claim 1, further comprising:
a coupling mechanism,
wherein the watertight housing comprises a top member and a bottom member, and
wherein the coupling mechanism couples the top member to the bottom member.

4. The electrical connector assembly of claim 3, wherein the coupling mechanism comprises at least one of:
a nut and bolt connection or a magnetic connection.

5. The electrical connector assembly of claim 4, wherein the magnetic connection comprises:
magnets coupled to the substrate.

6. The electrical connector assembly of claim 1, wherein the electrode on the substrate is electrically coupled to the coupling mechanism of the electrical connector assembly.

7. The electrical connector assembly of claim 1, the electrical connector further comprises:
a plug and socket connection.

8. The electrical connector assembly of claim 1, wherein the electrode on the substrate is disposed between the top member and the bottom member.

9. A method for conducting current to an electrode on a substrate, wherein the substrate is at least one of flexible or stretchable, the method comprising:
coupling an electrical connector assembly to the electrode on the substrate, the electrical connector assembly comprising:
a watertight housing; and
an electrical connector disposed in the watertight housing and capable of conducting about two amperes of current when connected to an external power source;
wherein the electrode on the substrate is at least one of in communication or coupled to the electrical connector.

10. The method of claim 9,
wherein the substrate includes a heating element; and
wherein the electrical connector assembly has at least one of an ambient temperature or a temperature below a temperature of the heating element during at least one of (i) a period of time of the external power source being in an operative state or (ii) use of the electrical connector assembly by a user.

11. The method of claim 9, wherein the electrical connector assembly comprises:
a coupling mechanism,
wherein the watertight housing comprises a top member and a bottom member, and
wherein the coupling mechanism couples the top member to the bottom member.

12. The method of claim 11, wherein the coupling mechanism comprises at least one of:
a nut and bolt connection or a magnetic connection.

13. The method of claim 12, wherein the magnetic connection comprises:
magnets coupled to the substrate.

14. The method of claim 9, wherein the electrode on the substrate is electrically coupled to the coupling mechanism of the electrical connector assembly.

15. The method of claim 9, wherein the electrical connector further comprises:
a plug and socket connection.

16. The method of claim 9, wherein the electrode on the substrate is disposed between the top member and the bottom member.

17. A system comprising:
a substrate, wherein the substrate is at least one of flexible or stretchable, the substrate further comprising an electrode;
an electrical connector assembly comprising:
a watertight housing; and
a conductive interface disposed in the watertight housing and capable of conducting about two amperes of current when connected to an external power source;
wherein the electrode on the substrate is at least one of in communication or coupled to the conductive interface.

18. The system of claim 17,
wherein the substrate includes a heating element; and
wherein the electrical connector assembly has at least one of an ambient temperature or a temperature below a temperature of the heating element during at least one of (i) a period of time of the external power source being in an operative state or (ii) use of the electrical connector assembly by a user.

19. The system of claim 17, wherein:
the electrical connector assembly further comprises a coupling mechanism,
the watertight housing comprises a top member and a bottom member,
the coupling mechanism couples the top member to the bottom member, and
the electrode on the substrate is electrically coupled to the coupling mechanism of the electrical connector assembly.

20. The system of claim 19, wherein the electrode on the substrate is disposed between the top member and the bottom member.
